(19) United States
(12) Patent Application Publication
(10) Pub. No.: US 2020/0230166 A1
(43) Pub. Date: Jul. 23, 2020

(54) Oligonucleotides in food, beverage, cosmetic and medicinal formulations

(71) Applicant: DNA Essence GmbH, Furigen (CH)

(72) Inventors: Johannes KABISCH, Muhltal (DE); Gunter FESTEL, Furigen (CH)

(21) Appl. No.: 15/781,357

(22) PCT Filed: Dec. 3, 2015
(86) PCT No.: PCT/EP2015/078484

§ 371 Date: Jun. 4, 2018

(51) Int. Cl. A61K 31/7088; A61K 8/60; A61K 9/00; A61Q 19/00

(52) U.S. Cl. CPC A61K 31/7088; A61K 8/606; A61Q 19/00; A61K 9/0095; A61K 9/0014

(57) ABSTRACT

The present invention relates to formulations comprising oligonucleotides of mammalian, avian, reptilian or plant, non-human DNA. The formulations may be food, beverage food, cosmetic or medicinal formulations. In one aspect, the oligonucleotides are present in the form of DNA-carrier complexes. Further provided are a kit comprising a formulation ac cording to the invention and a method for producing the formulations of the invention. It is also envisaged that the formulations are used in personal lubricants or spice mixtures.

This invention relates to formulations comprising oligonucleotides, in particular to food, cosmetic and medicinal formulations. The used oligonucleotides originate from wildlife animals and plants, but are obtained via molecular biological means; thus, the invention will help to conserve these animals and plants. In one aspect of the invention, oligonucleotides are added in the form of DNA carrier complexes. Another aspect of the invention is directed at methods of producing the food, cosmetic and medicinal formulations comprising the aforementioned oligonucleotides.

Many components used in traditional Chinese medicine, which knows over 6000 different ingredients, are taken directly from natural occurring sources, be it minerals, animals, plants or herbs. These extracts can cure diseases, promote longevity and improve general health and well-being. Thanks to a longstanding medical tradition the effect of these ingredients has been well proven and documented. However, following industrialization and the spread of traditional Chinese medicine all over the world, the extensive use of natural resources has brought some of the species from which the extracts are taken to the brink of extinction.

In recent years, biologists, chemists and other researches have directed their efforts to deciphering and annotating the genomes of a multitude of animal and plant species. This has led to the discovery of many novel gene sequences, among which are also many genes found in animals and plants that are used in traditional Chinese medicine. With the help of homology analysis in combination with activity assays and cell studies, it is possible to attribute a function to some of these newly discovered genes.

These novel findings and discoveries can then be combined with the knowledge of traditional Chinese medicine to identify a connection between the observed effect and possible candidate genes that can subsequently be isolated or produced in vitro. Not only will this help to provide a larger patient group with useful natural ingredients, but at the same time, the present invention will contribute to the protection of endangered wildlife, as it offers a possibility to obtain the necessary ingredients by means other than killing animals.

For example, various parts of the tiger are popular for the preparation of Chinese pharmacopeia because of their purported anti-inflammatory effects as well as their healing properties in connection with ulcer, rheumatic pain and burns. The regenerative organs of the tiger, in particular the penis, are prescribed as an aphrodisiac. Possible candidate genes and genomic regions that will provide a similar effect are the growth hormone 1 (GH1), the sex determining region of the Y chromosome (SRY) and a gene involved in the final steps of testosterone synthesis (HSD17B3) of Panthera tigris altaica (tiger), which have been identified as the sequences depicted in SEQ ID NO:1, 2 and 3, respectively.

The horn of the rhinoceros is used to treat fevers, convulsions and gout; in some countries, the horn is also used as an aphrodisiac. The main component of the rhinoceros horn is keratin, which is encoded by structural keratin type II (HIY3) of Buceros rhinoceros silvestris (rhinoceros) (Hieronymus T L, Witmer L M, Ridgely R C. (2006) Structure of white rhinoceros (Ceratotherium simum) horn investigated by X-ray computed tomography and histology with implications for growth and external form. J Morphol. 267(10):1172-6.); cf. SEQ ID NO:4.

Shark fins are considered to boost sexual activity, promote energy, lower cholesterol levels and rejuvenate the body. The collagen type XIII alphal structural protein of Callorhynchus milli (shark) has been identified as depicted in SEQ ID NO:5. Collagen is the main component of shark fins (Motta P J. (1977) Anatomy and Functional Morphology of Dermal Collagen Fibers in Sharks. Copeia. 3:454-464).

Bamboo extracts are found useful in the treatment of epilepsy, febrile conditions and vomiting. Furthermore, bamboo is known for its rapid growth, a trait that is associated with small auxin up RNA 8 as seen in SEQ ID NO:18 (Peng Z, Zhang C et al. (2013) Transcriptome Sequencing and Analysis of the Fast Growing Shoots of Moso Bamboo (Phyllostachys edulis). PLoS ONE. 8(11):e78944. doi:10.1371/journal.pone.0078944.).

Another aspect of Chinese culture are the 12 animal signs. Each of these zodiacs is associated with specific character traits; the annotation of the genomes of the respective species enables scientists to associate these traits with particular genes.

For example, the rat is known for its ambition; this trait is related to the 3 beta-hydroxysteroid dehydrogenase, which is essential for steroid hormone formation; cf. SEQ ID NO:6 (Lachance Y, Luu-The V et al. (1990) Characterization of human 3 beta-hydroxysteroid dehydrogenase/delta 5-delta 4-isomerase gene and its expression in mammalian cells. J Biol Chem. 265(33):20469-75.).

In sheep, the oxytocin receptor (OXTR), which is related to empathy, has been identified as the sequence of SEQ ID NO:7 (Tost H, Kolachana B et al. (2010) A common allele in the oxytocin receptor gene (OXTR) impacts prosocial temperament and human hypothalamic-limbic structure and function. Proc Natl Acad Sci U S A. 107(31):13936-41.doi: 10.1073/pnas.1003296107.).

In ape, the formin-binding protein I (FNBP1L), which is associated with intelligence, has been identified as the sequence depicted in SEQ ID NO:8 (Benyamin B, Pourcain B et al. (2014) Childhood intelligence is heritable, highly polygenic and associated with FNBP1L. Molecular psychiatry. 19(2):253-258. doi:10.1038/mp.2012.184.).

In pig, oxytocin, which is also related to generosity, has been described as the sequence of SEQ ID NO:9 (Barraza J A, Zak P J. (2009) Empathy toward strangers triggers oxytocin release and subsequent generosity. Ann N Y Acad Sci. 1167:182-9. doi: 10.1111/j.1749-6632.2009.04504.x).

The rooster is valued for executive functioning, which is related to the gene WDR72; cf. SEQ ID NO:10 (LeBlanc M, Kulle B et al. (2012) Genome-wide study identifies PTPRO and WDR72 and FOXQ1-SUMO1P1 interaction associated with neurocognitive function. J Psychiatr Res. 46(2):271-8. doi: 10.1016/j.jpsychires.2011.11.001.).

The dragon is known for intelligence; in its closest living relative, the Varanus comodovarensis, brain plasticity can be traced back to the gene BDNF as seen in SEQ ID NO:11 (Binder D K, Scharfman H E. (2004) Brain-derived Neurotrophic Factor. Growth factors (Chur, Switzerland). 22(3):123-131. doi:10.1080/08977190410001723308.).

The Chinese zodiac rabbit possesses creativity, which is based on neuregulin 1 as seen in SEQ ID NO:12 (Kéri S. (2009) Genes for psychosis and creativity: a promoter polymorphism of the neuregulin 1 gene is related to creativity in people with high intellectual achievement. Psychol Sci. 20(9):1070-3. doi: 10.1111/j.1467-9280.2009.02398.x.)

The snake is known to strike quickly, a trait that is related to the gene encoding acetylcholinesterase (AChE). AChE promotes nerve functioning and development and is encoded in the snake by SEQ ID NO:13 (Sternfeld M, Ming G et al. (1998) Acetylcholinesterase enhances neurite growth and synapse development through alternative contributions of its hydrolytic capacity, core protein, and variable C termini. J Neurosci. 18(4):1240-9.).

The dog is cherished for loyality, a trait that is related to oxycytocin (Scheele, D. et al. (2012) Oxytocin Modulates Social Distance between Males and Females. J. Neurosci. 32: 16074-16079.). The sequence of the corresponding oxytocin receptor is shown in SEQ ID NO:14.

The buffalo's trait of perseverance and the ability to focus on one task is related to the formation of 2-phenylethylamine, which is controlled by dopamine beta-hydroxlase Orsfeld M, Spadafore M, Prüβ BM. (2013) 13-phenylethylamine, a small molecule with a large impact. WebmedCentral. 4(9):4409.); cf. SEQ ID NO:15.

The horse is known to seek novelty and to be curious; this is connected to the dopamine receptor D4 (Munafò, M. R. et al. (2008) Association of the Dopamine D4 Receptor (DRD4) Gene and Approach-Related Personality Traits: Meta-Analysis and New Data. Biological Psychiatry 63:197-206), which can be seen in SEQ ID NO:16.

Another animal known for its outstanding qualities in particular with regard to eye sight is the eagle. Here, the relevant gene is crystallin beta, which is a component of the eye lense and is depicted in SEQ ID NO:17.

While all of the above mentioned sequences have been deciphered and annotated, their effect on the human body has not been elucidated so far. In fact, prior to the invention, introduction of particular gene sequences from any origin other than human into humans has not been considered. Thus, it is an objective of the present invention to provide active ingredients of animal and plant extracts in the form of isolated mammalian, reptilian, avian and plant, non-human gene sequences for the administration to humans. By providing the active ingredients of known traditional formulations in the form of in vitro produced oligonucleotides, killing of animals is circumvented and animal wildlife thus protected.

Furthermore, using whole animals or parts thereof poses a risk since it involves uptake of the complete animal or plant genome, which might harbor viral sequences. Additionally, the extract might be contaminated with pathogens. It has been shown that such viruses and pathogens are involved in the development of cancers (Burnett-Hartman A N, Newcomb P A et al. (2008) Infectious Agents and Colorectal Cancer: A Review of Helicobacter pylori, Streptococcus bovis, JC Virus, and Human Papillomavirus. Cancer Epidemiol Biomarkers Prey. 17: 2970-2979. doi: 10.1158/1055-9965.EPI-08-0571). Thus, providing defined, synthetic, virus-free oligonucleotides ensures a high level of medical safety and can protect individuals from harmful agents.

Previously, DNA spheres containing DNA probes of around 100 nucleotides have been described (U.S. Pat. No. 8,835,178 B2). These probes are used in scientific setups as test reagent for atmospheric release and bio-hazard detection; in contrast to the present invention, the DNA within the spheres is not expressed, but merely detected via PCR.

Other systems have been coined for the delivery and expression of nucleic acid molecules (U.S. Pat. No. 7,030,097 B1). This system consists of a polymeric structure formed by a biocompatible polymer and a mixture of nucleic acid molecules and a co-dispersant. Using this system, DNA can be delivered into an organism, but only with the help of additional polymers, thus complicating the production process and additionally introducing foreign structures that might elicit immune responses.

Until recently, it was believed that delivery of “naked” DNA, i.e. DNA without any uptake promoting agents, does not result in efficient levels of DNA expression (Villemejane J, Mir L M. (2009) Physical methods of nucleic acid transfer. Brit J Pharma. 157: 207-219.).

However, it has now been shown that complete genes may be taken up via the digestive tract (Spisák S, Solymosi N et al. (2013) Complete Genes May Pass from Food to Human Blood. PLoS ONE 8(7): e69805. doi: 10.1371/journal.pone.0069805). Additionally it has been shown that the bacterial communities inhabiting the human gut can take up DNA and express these genes as functional products (Hehemann, J H et al. (2010) Transfer of carbohydrate-active enzymes from marine bacteria to Japanese gut microbiota. Nature 464: 908-91. doi: 10.1038/nature08937).

Accordingly, it is an object of the present invention to provide the respective genes from animal sources in a formulation that allows for expression of these genes and not just delivery in a human individual, while at the same time refraining from using any superfluous carrier polymers.

The object is solved by a formulation comprising at least 1 million oligonucleotides of mammalian, avian, reptilian or plant, non-human DNA, which oligonucleotides have sequences that are at least 80% homologous to one or more of the sequences selected from the group consisting of SEQ ID NOs: 1 to 18.

Throughout the specification, the term “oligonucleotide” is to be conceived as comprising single-stranded or double-stranded oligonucleotides or a mix thereof. The oligonucleotides may also consist of RNA. Each oligonucleotide may have a length of 10 to 2500 bp and may also be a concatemer of the same oligonucleotide in repetition.

In the present specification, the terms “homology” and “homologous” are meant to be understood as sequence identity. Homology between amino acid sequences or nucleotide sequences can be examined using the algorithm BLAST (Altschul, S F, Gish W et al. Basic local alignment search tool. (1990) J. Mol. Biol. 215:403-410.) and its further developed versions BLASTN and BLASTX. Default parameters should be employed for all three programs.

Throughout this specification and the claims which follow, unless the context requires otherwise, the word “comprise” and variations such as “comprises” and “comprising” will be understood to imply in inclusion of a stated integer or step or group of integers but not the exclusion of any other integer or step or group of integers or steps. As used in this specification and the appended claims, the singular forms “a”, “an” and “the” include plural referents, unless the context clearly dictates otherwise.

Surprisingly it was found that the use of 1 million oligonucleotides is more efficient than the uptake of the related extract in terms of promoting the expression of the relevant gene. This is most likely due to the high copy number of the respective genes in comparison with only small amounts of the respective gene sequences that are present in traditionally prepared extracts.

Within the scope of the invention, it is envisaged that the formulation may comprise 1, 1.5, 2, 2.5, 3, 3.5, 4, 4.5, 5, 6, 7, 8, 9 or 10 million oligonucleotides. Preferably, the formulation of the invention comprises 1, 2 or 3 million oligonucleotides; most preferably, the formulation of the invention comprises 1 million oligonucleotides.

The oligonucleotides can have a sequence homologous to one or more of the sequences selected from the group consisting of SEQ ID NOs: 1 to 18, so that different types of oligonucleotides are present in the formulation. For example, the one or more sequences of the oligonucleotide can be at least two, at least three, at least four or at least five different sequences. In embodiments where two or more different types of oligonucleotides are present in the formulation, the formulation comprises 1 million copies of each of the different oligonucleotides. It is envisaged that in embodiments comprising oligonucleotides with two or more different sequences, the different sequences are from genes likely to show a similar effect; e.g., in one embodiment, oligonucleotides with SEQ ID NOs: 1, 2 and 3 or oligonucleotides with SEQ ID NOs: 1, 2, 3 and 4 are combined. In embodiments where genes with a similar effect are combined the overall effect on the individual will be even greater than if each type of oligonucleotide is taken separately.

Oligonucleotides used in the formulation of the present invention can have 80% or more homology to SEQ ID NOs: 1 to 18. In a preferred embodiment, the oligonucleotides have more than 90% homology to SEQ ID NOs: 1 to 18. In the most preferred embodiment, the oligonucleotides have more than 95% homology to SEQ ID NOs: 1 to 18. It is further envisaged that the oligonucleotides might contain “silent” mutations. A “silent mutation” is hereby defined as a change of the nucleotide sequence that, due to the ambiguity of the genetic code, does not result in a change of the amino acid sequence upon expression. Silent mutations may be introduced because of differences in codon usage between different species, i.e. frequency of the occurrence of synonymous base triplets all encoding for the same amino acid.

To practice the present invention, unless otherwise indicated, conventional methods of chemistry, biochemistry, cell biology and recombinant DNA techniques are employed which are explained in the literature in the field (cf., e.g., Molecular Cloning: A Laboratory Manual, 4^{th} Edition, Green and Sambrook eds., Cold Spring Harbor Laboratory Press, Cold Spring Harbor 2012).

The oligonucleotides of the invention may be obtained by all methods generally applied in the field, e.g., polymerase chain reaction using genomic DNA or plasmid DNA as a template, reproduction in bacteria or cells or in silico synthesis. When oligonucleotides are expressed in recombinant systems, the skilled person is capable of designing a suitable system including an appropriate promoter and plasmid vector as well as a cell line or bacterial strain that can express the oligonucleotide used in the formulation of the invention. For example, the plasmid constructs used to this extent may comprise a suitable promoter with high activity in the respective cell line or bacterial strain and further transcription and translation regulators as well as suitable linkers with appropriate restriction sites. The skilled person is also aware of the influence of culture conditions on plasmid yield.

If necessary, produced oligonucleotides can be purified by precipitation, with the help of commercially available purification kits (e.g. Wizard® PCR Preps DNA Purification System, Promega, Madison, Wis., USA) or via agarose gel electrophoresis. The person skilled in the art is well aware of how each of these methods can be optimized to maximize oligonucleotide yield and purity.

In order to ensure long-term integrity and stability of the oligonucleotides within the formulation, additional agents inhibiting the degradation of the oligonucleotides can be added to the formulation. These include DNase inhibitors in the form of specific proteins, nucleotides, anthracycline and aminoglycoside antibiotics, synthetic organic and inorganic compounds or commercially available DNase removing agents (Ambion® DNase Removal Reagents, Life Technologies, Carlsbad, Calif., USA).

The oligonucleotides may also be modified themselves to increase their stability. Possible modifications include phosphorothiorate, methylphosphonate, phosphoroamidate, boranophosphate, phosphoroacetate or 2′,5′-phosphodiester. Amide-linked oligonucleotides, morpholino and peptide nucleic acids are also contemplated in the scope of the invention.

Furthermore, the formulation of the invention may also comprise cellular uptake agents. Cellular uptake agents are molecules, ligands or moieties that facilitate uptake of the oligonucleotide into cells. The cellular uptake agents can be independent of the oligonucleotides or fused to one or more of them. One group of cellular uptake agents includes lipids and cationic lipids. Another group of cellular uptake agents are receptor binding ligands that can recognize surface molecules on cells. In general, all molecules that target markers expressed on the surface are contemplated as cellular uptake agents; in particular, growth factors, cytokines and antibodies may be used. Fragments of suitable ligands may also be used in the formulations and methods of the present invention.

The cellular uptake agents can be covalently or non-covalently linked to the oligonucleotides. Covalent linkage can be based on UV crosslinking or chemical crosslinking, whereas non-covalent attachment can be achieved by exploiting electrostatic or hydrophobic interactions. All of these methods have been previously described and are well known to the skilled artisan.

The formulation of the invention can be administered alone or in combination with a physiologically acceptable carrier, e.g. solvents, perfumes or colorants, sunscreens or other ingredients for medicinal or cosmetic use. Furthermore, vehicles such as water, saline or other delivery vehicles can be added to the formulation. It is also envisaged that the formulation of the present invention is mixed with commercially available “energy” drinks such as RedBull®, Monster Energy®, Rockstar®, NOS® or Amp Energy®.

In another aspect of the invention, the formulation contains the oligonucleotides in the form of DNA carrier complexes comprising oligonucleotides and a physiologically acceptable stabilizer. This results in the oligonucleotides being protected from nuclease activity after application or administration and thus, in an increase of the number of intact oligonucleotides that can be expressed within the cells of the individual. Because a higher percentage of the oligonucleotides remain intact for a longer time, the use of a DNA carrier complex makes it possible to reduce the overall copy number of oligonucleotides.

The person skilled in the art is well aware how such DNA carrier complexes can be formed and how to choose a suitable stabilizer material. Physically acceptable stabilizer include sodium or potassium alginate, hyaluronic acid, poly(ethylene-co-vinyl acetate), poly-L-lactide, poly-D-lactide, polyglycolide, poly(lactide-co-glycolide), polyanhydride, polyorthoester, polyphosphazene, proteinaceous polymer, polyester, silicone, or combinations thereof.

Depending on the mode of application, the DNA carrier complexes can be appropriately sized in the range between 5 and 500 nm.

The DNA carrier complexes may also comprise cellular uptake agents, which can be covalently linked or non-covalently attached to the DNA carrier complex.

In another embodiment, the present invention relates to a cosmetic formulation or a topical dermatological formulation. The cosmetic formulation of the present invention allows for topical application of the oligonucleotides, resulting in local expression thereof. This leads to an increased effect in particular regions of the body, whereas no side effects are observed in other body parts. Cosmetic formulations according to the present invention may comprise preservatives such as esters of p-hydroxybenzoic acid, e.g. methyl- p-hydroxybenzoate, and propyl p-hydroxybenzoate; cis-1-(3-chloroallyl)-3,5,7-triaza-1-azoniaadamantane chloride; ethylenediaminetetraacetic acid (EDTA) and salts of EDTA; imidazolidinyl urea; sodium N-lauryl-β-iminodipropionate and the like or any combination thereof. Additionally, color and essence can be added to the cosmetic formulation of the present invention. Color additives include both natural and artificial dyes, e.g., carotenoid derivatives and iron oxides, while essences can include any non-irritating natural and artificial oils and perfumes. In general, the individual ingredients used in the formulation should be of a quality or purity suitable for cosmetic use and the cosmetic formulation will be non-stinging and non-irritant.

In yet another embodiment, the present invention relates to a food formulation and a beverage food formulation. Food and beverage food formulations of the present invention enable the convenient uptake of a large amount of the respective oligonucleotides and are particularly useful when the desired effect is not limited to specific body parts. They may additionally comprise natural and artificial sweeteners such as sugar, mannitol, sorbitol, xylitol, lactitol, isomalt, maltitol and hydrogenated starch hydrolysates (HSH); salt; flavor enhancers; acidifiers; electrolytes; and emulgators. They may also comprise whole grains, nuts, protein sources, seeds, dried fruits or other nutrients.

Furthermore, it is envisaged that the beverage food formulation of the present invention comprises stimulating agents such as caffeine, guarana or taurine and additional ingredients such as herbal extracts from, e.g., green tea, ginseng and gingko biloba, as well as vitamins such as B vitamins and sugar. Combination of the beverage food formulation of the present invention taken with stimulating agents further enhances the effect of the oligonucleotides, in particular where this effect is connected to male potency.

The beverage food formulation of the invention may be premixed with the oligonucleotides or the oligonucleotides may be contained in the beverage food formulation as a separate entity or may be provided to the customer in a form that allows for ad hoc mixture of the beverage food formulation. If the oligonucleotides are present as a separate entity, they may be contained in a capsule, a gelous ball (e.g. comprising tapioca) or a pill within the beverage food formulation. If the oligonucleotides are provided in a form allowing for ad hoc mixture by the customer himself, they may be, for example, provided within a sachet, a teabag, an injector or a dispenser that is provided together with a conventional beverage container such as a can or a bottle. It is also envisaged that the injector or dispenser is directly connected to the food beverage container.

In one embodiment, the present invention relates to a medicinal formulation. The medicinal formulation of the present invention may additionally comprise a suitable pharmaceutical excipient such as starch, glucose, lactose, sucrose, gelatin, malt, rice, flour, chalk, silica gel, sodium stereate, glycerol monostereate, talc, sodium chloride, dried skim milk, glycerol, propylene, glycol, water, ethanol and the like.

In further embodiments, the present invention relates to formulations, wherein the sequence of the oligonucleotides is SEQ ID NO:1, 2, 3, 4, 5, 6, 7, 8, 9, 10, 11, 12, 13, 14, 15,16,17 or 18.

In one embodiment of the invention the oligonucleotides have the sequence of SEQ ID NO:1. Application or administration of this formulation leads to increased potency, stimulates libido and improves sexual performance, particularly in male individuals.

In another embodiment of the invention the oligonucleotides have the sequence of SEQ ID NO:2. Application or administration of this formulation leads to increased potency, stimulates libido and improves sexual performance, particularly in male individuals.

In yet another embodiment of the invention, the oligonucleotides have the sequence of SEQ ID NO:3. Application or administration of this formulation leads to increased potency, stimulates libido and improves sexual performance, particularly in male individuals.

In another embodiment the oligonucleotides have the sequence of SEQ ID NO:4. Application or administration of this formulation helps with febrile conditions and convulsions and also stimulates sexual activity.

In a further embodiment the oligonucleotides have the sequence of SEQ ID NO:5. Application or administration of this formulation improves the sexual appetite, boosts energy levels and helps to rejuvenate the body.

In another embodiment, the oligonucleotides have the sequence of SEQ ID NO:6. Application or administration of this formulation leads to increased steroid hormone expression, resulting in increased ambition in the individual.

In another embodiment, the oligonucleotides have the sequence of SEQ ID NO:7. Application or administration of this formulation leads to higher levels of the oxytocin receptor, which impacts social behavior.

In another embodiment, the oligonucleotides have the sequence of SEQ ID NO:8. Application or administration of this formulation leads to higher expression levels of formin-binding protein 1-like protein and thus modulates brain development and activity.

In another embodiment, the oligonucleotides have the sequence of SEQ ID NO:9. Application or administration of this formulation leads to increased production of oxytocin, which influences social bonding and interaction.

In another embodiment, the oligonucleotides have the sequence of SEQ ID NO:10.

Application or administration of this formulation leads to higher expression levels of WDR72, which promotes neurocognitive functioning.

In another embodiment, the oligonucleotides have the sequence of SEQ ID NO:11. Application or administration of this formulation leads to increased expression of brain-derived neutrophic factor and, thus, enhances brain plasticity.

In another embodiment, the oligonucleotides have the sequence of SEQ ID NO:12. Application or administration of this formulation leads to increased levels of neuregulin 1, promoting creativity.

In another embodiment, the oligonucleotides have the sequence of SEQ ID NO:13. Application or administration of this formulation results in overexpression of AChE, which positively influences nerve functioning and development.

In another embodiment, the oligonucleotides have the sequence of SEQ ID NO:14. Application or administration of this formulation results in expression of OXTR, which will increase empathy in individuals.

In another embodiment, the oligonucleotides have the sequence of SEQ ID NO:15. Application or administration of this formulation increases expression of dopamine beta-hydroxlase and therefore improves power of concentration.

In another embodiment, the oligonucleotides have the sequence of SEQ ID NO:16. Application or administration of this formulation leads to enhanced expression of the dopamine receptor D4, making individuals more likely to take risks.

In another embodiment, the oligonucleotides have the sequence of SEQ ID NO:17. Application or administration of this formulation leads to higher levels of crystalline beta, thus improving eye-sight.

In another embodiment, the oligonucleotides have the sequence of SEQ ID NO:18. Application or administration of this formulation will promote growth.

Furthermore, it is envisaged to use the formulation of the invention in one embodiment in personal lubricants. These lubricants can be water-based, oil-based or silicone-based. A sufficient amount of the formulation of the invention is added so that 1 ml contains at least 1 million oligonucleotides. The personal lubricant in which the formulation of the invention is used may contain any of the ingredients compatible with a personal lubricant such as buffer salts, clouding agents, anticaking agents, flavors, inorganic base, water-soluble cellulose-derived polymers, polyhydric alcohol and color.

The personal lubricant comprising the formulation of the invention may be prepared conventionally or be adapted to specific needs at the manufacturing site. Conventional preparation consist of mixing all the required ingredients by dissolving them in water of appropriate purity and adding the rehydrated oligonucleotide in varying concentrations for example from 1 million to 10 million oligonucleotide molecules per ml of personal lubricant, depending on the desired strength. The oligonucleotide formulation of the invention may be added by dripping the rehydrated oligonucleotide solution into the lubricant solution, followed by a mixing step.

In yet another embodiment, the present invention relates to a method to produce the formulations of the present invention.

Lyophilized oligonucleotides of SEQ ID NOs:1-18 can be rehydrated with an appropriate aqueous buffer system such as monopotassium phosphate (E340) or potassium tartrate (E336) buffer adjusted to pH 8, in order to reduce depurination of the oligonucleotide. The resulting solution can be added to yield the formulations of the invention.

As a general means of preparation suitable for most applications, rehydrated oligonucleotides can simply be added as a solution to a certain final concentration, which is dependent on the desired amount of molecules. The amount of molecules is determined as follows: oligonucleotides are quantified by spectroscopic means (e.g. NanoDrop, ThermoFischer, USA) at a wavelength of 260 nm. The result, an amount expressed in nanogram/μl is converted via the following formula: oligonucleotide concentration (ng/μl)/[0.66*DNA size (bp)] for double stranded oligonucleotides and oligonucleotide concentration(ng/μl) [0.33*DNA size (bp)] for single stranded oligonucleotides, respectively.

Lyophilized oligonucleotides may be mixed with dry or liquid flavouring and/or colouring agents in and may be contained in a container.

The invention also relates to a kit for individual preparation of the formulation of the invention by the end consumer. This kit comprises containers with different sets of lyophilized oligonucleotides, an appropriate buffer system, a measuring device and instructions on how to prepare different strength of the final product and mixing formulations with foods, beverages, medicinal formulation, cosmetic formulation or a topical dermatological formulation.

In another formulation the formulation of the invention can be used in a spice mixture.

It is to be understood that the present invention is not limited to the particular methodology, protocols and reagents described herein, as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meaning as commonly understood by one of ordinary skill in the art.

The invention is furthermore explained by the following figures, which are, however, not intended to limit the scope of the invention.

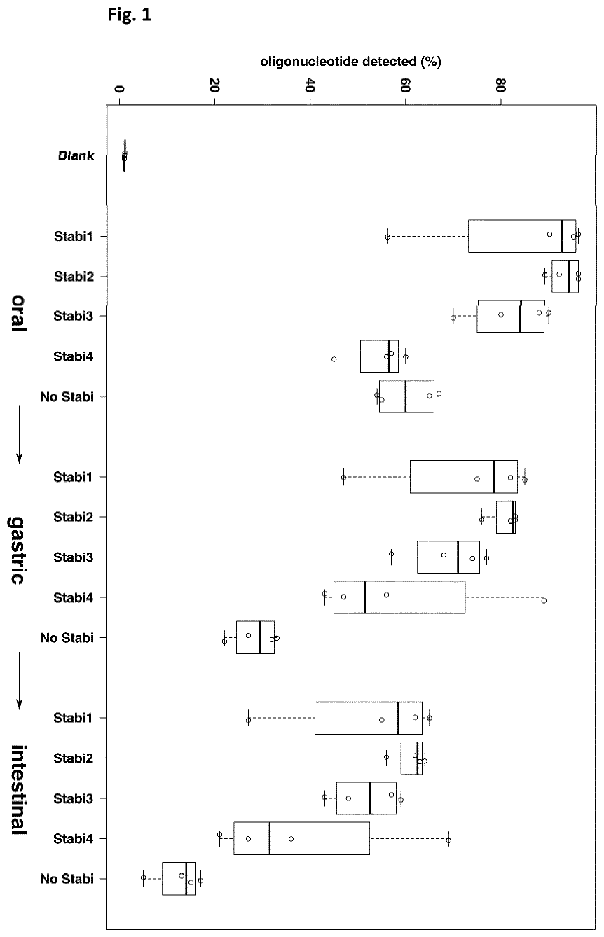

FIG. 1 shows the effect of different stabilizers on digestion of the oligonucleotides of the invention during various phases of in vitro digestion. Blank: control with buffer only.

The following examples are intended to illustrate the invention. They shall not limit the scope of the claims in any way.

### Example 1

### Obtaining DNA Sequences through DNA Sequencing

DNA is extracted from body-fluids or tissues of the animal or plant of interest using commercially available kits such as Quick-DNA™ Universal Kit (Zymo Research Europe GmbH, Freiburg, Germany). Shotgun libraries for sequencing are generated using the Nextera XT DNA Sample preparation kit (Illumina, San Diego, USA) following the manufacturer instructions. The whole genome of the corresponding animal or plant is sequenced with the Genome Analyzer IIx (Illumina, San Diego, USA). The libraries are sequenced in a 112 bp paired end single indexed run. The resulting paired end reads are assembled using SPAdes assembler (Bankevich A et al. (2012) SPAdes: A New Genome Assembly Algorithm and Its Applications to Single-Cell Sequencing, J Comp Biol. 5: 455-77, doi:10.1089/cmb.2012.0021) or MIRA (Chevreux B, Wetter T et al. Genome Sequence

Assembly Using Trace Signals and Additional Sequence Information. in German Conference on Bioinformatics, 1999, 45-56.) on an appropriately equipped GNU/Linux computing cluster. In order to identify homologs of human genes with a known function, the contiguous sequences (contigs) are searched with tblastn (E. Michael Gertz et al. Composition-Based Statistics and Translated Nucleotide Searches: Improving the TBLASTN Module of BLAST. BMC Biol. 2006, Dec. 7; no. 1:41, doi:10.1186/1741-7007-4-41.) using the human protein sequence.

### Example 2

### Production of Specific Oligonucleotide Molecules

The oligonucleotides comprising the sequence of interest (SOI), i.e., SEQ ID NOs: 1 to 18, can be produced by several means:

### 1. Polymerase Chain Reaction using a Thermal Cycler

Polymerase chain reaction (PCR) is performed according to the method described previously (Saiki RK et al. Enzymatic Amplification of Beta-Globin Genomic Sequences and Restriction Site Analysis for Diagnosis of Sickle Cell Anemia. Science 1985, Dec. 20. 230, no. 4732: 1350-54, doi:10.1126/science.2999980.). For the amplification of each sequence of interest (SOI) oligonucleotide primers with a length between 20 and 25 bases and always ending in a guanine or cytosine base are chemically synthesized and 100 pmol of each primer are added to 1 μg of genomic DNA template or a plasmid containing a chemically synthesized DNA (Genscript,USA) containing the SOI. 35 amplification cycles are performed in a thermocylcer (DNA Engine Tetrad2, Bio-Rad, USA) using the polymerase chain reaction (PCR) according to the instructions of the manufacturers (OptiTaq, RoboKlon, Germany). Denaturation is performed at 98° C. for 30 seconds, primer annealing at 55° C. for 25 seconds and primer extension at 72° C. for 1 min per kilobase of the SOI. An analytic sample (approx. 2 μI) of the resulting product is analyzed on a 1.0% agarose gel (CAS-Nr. 9012-36-6) with a DNA-size standard (1 kbp DNA-Ladder, Carl-Roth GmbH+Co. KG, Germany). After staining of the agarose gel (Roti-GelStain, Carl-Roth GmbH+Co. KG, Germany), presence of oligonucleotide of the expected size of the SOI is confirmed on a transilluminator (Benchtop UV Transilluminators, UVP, USA). If unspecific bands other than that of the SOI are detected, PCR is repeated and optimized by varying the annealing temperature, adding enhancers such as PCR-grade DMSO (CAS-Nr. 67-68-5) or by varying buffer compositions and concentration of the utilized template DNA. Upon establishing a highly specific protocol that only amplifies the SOI, upscaling is achieved by increasing the amount of oligonucleotides and deoxynucleotides used and parallelizing the process in 96- or 384 well PCR plates (twin.tec PCR plates, Eppendorf, Germany).

### 1.1 Product Purification

The resulting PCR product has to be separated from the DNA polymerase, the buffer components and deoxynucleotides. This is achieved by either using commercially available purification kits (e.g. NucleoSpin Gel and PCR Clean-up, MACHEREY-NAGEL, Germany) following the instructions of the supplier or by means of precipitation. For precipitation, the PCR reactions are pooled and sodium acetate (CAS-Nr. 127-09-3) (0.3 M, pH 5.2, final concentration) is added to the oligonucleotide mix followed by 2 volumes of ice cold absolute ethanol (CAS-Nr. 64-17-5) on 1 volume oligonucleotide, followed by an incubation at −80° C. for at least two hours. This precipitation is followed by a 30 minute centrifugation at 17000×g. After removing the supernatant, the oligonucleotide pellet is washed with 70% ethanol, centrifuged for 15 minutes at 17000×g, air dried for 30 minutes and resuspended with 2.0 M NaCl (7647-14-5) and 50 mM Tris-CI (CAS-Nr. 1185-53-1). pH is adjusted to 7.5 with HCl (CAS-Nr. 7647-01-0).

### 1.2 Storage

The resulting product is mixed with sucrose (CAS-Nr. 57-50-1) to a final concentration of 20 mg/μl and filled into brown hydrolytic class 1 Type FIOLAX glass vials (Schott, Germany) which are closed with butyl rubber lyophilisation stoppers (Wheaton, USA). Lyophilisation is performed as follows: vials are frozen to −21° C., drying is started at a shelf temperature of −21° C. and a chamber pressure of 0.35 mbar and the temperature elevated to −10° C. during the drying process. Secondary drying is carried out at a shelf temperature of −21° C. and chamber pressure of 0.2 mbar. The resulting product is sealed with temper proof caps (West Flip-Off seals, West Pharmaceutical, Germany). Lyophilised DNA can be stored at 25° C./60% relative humidity (6 months), 2-8° C. (24 months), and −20° C. (66 months) in the dark without significant degradation (van der Heijden I, Beijnen J H et al. (2013) Long Term Stability of Lyophilized Plasmid DNA pDERMATT. Int J Pharm. 10; 453, no. 2: 648-50, doi:10.1016/j.ijpharm.2013.06.010.).

### 2. Replication in Microbial Cells

Specific oligonucleotide sequences can be produced in large quantities by integrating them into a replicative plasmid, which can be used to transform bacterial cells, such as Escherichia coli and Bacillus subtilis, which, while propagating and dividing, will replicate and propagate the plasmids carrying the SOI.

In order to yield economically feasible amounts of oligonucleotide, high copy numbers per cell, high cell densities and efficient downstream processing methods must be achieved.

The SOI can be combined with a replicative plasmid using standard cloning techniques (e.g. SLIC as described previously (Kumpfmüller J, Kabisch J et al. (2013) An Optimized Technique for Rapid Genome Modifications of Bacillus Subtilis. J Microbiol Met. 3: 350-52, doi:10.1016/j.mimet.2013.10.003)) with an origin of replication (ORI) resulting in high-copy numbers in the cells. Examples for such origins are the ColE1 and pUC ORIs (Prather K J et al. (2013) Industrial Scale Production of Plasmid DNA for Vaccine and Gene Therapy: Plasmid Design, Production, and Purification. Enz Micro Tech. 7: 865-83, doi:10.1016/S0141-0229(03)00205-9.), or origins with so called “runaway-replication”, which, after a temperature shift, lose control of replication regulation (Uhlin B E and Nordstrom K A. (1978) Runaway-Replication Mutant of Plasmid R1drd-19: Temperature-Dependent Loss of Copy Number Control. Mol General Gen MGG. 165, no. 2: 167-79, doi:10.1007/BF00269904; Remaut E, Tsa H. (1983) Improved Plasmid Vectors with a Thermoinducible Expression and Temperature-Regulated Runaway Replication. Gene. no. 1: 103-13.) and can lead to copy numbers of 1000 per cell for the Gram-negative bacterium Escherichia coli. For the Gram-positive bacterium Bacillus subtilis the RepE ORI (Swinfield T J et al. (1990) Physical Characterisation of the Replication Region of the Streptococcus Faecalis Plasmid pAM Beta 1. Gene. 87, no. 1: 79-90.) can be applied. The resulting plasmid carrying the SOI has to be transformed into the appropriate host. Transformation, meaning the transfer of plasmid DNA into bacterial cells, can be achieved through electroporation of B. subtilis (Kabisch J et al. (2012) Characterization and Optimization of Bacillus Subtilis ATCC 6051 as an Expression Host. J Biotechnol. doi:10.1016/j.jbiotec.2012.06.034.) and E. coli (Dower W J, Miller J F et al. (1988) High Efficiency Transformation of E. coli by High Voltage Electroporation. Nuc Acids Res. 16, no. 13: 6127-45, doi:10.1093/nar/16.13.6127.) following the protocols in the indicated publications.

The cells harboring the plasmids are cultivated in an incubator shaker using a C-source release medium such as Enpresso (BioSilta Ltd., Cambridgeshire, United Kingdom) or in a fed-batch process using a bioreactor. Several optimized processes have been described for the most commonly applied E. coli strains (Quaak SGL et al. (2008) GMP Production of pDERMATT for Vaccination against Melanoma in a Phase I Clinical Trial. Euro J Pharma Biopharma. 70, no. 2: 429-38, doi:10.1016/j.ejpb.2008.05.002.). In short, lysogeny-broth (LB) plates, containing an appropriate antibiotic for keeping selection pressure on plasmid carrying cells, are generously inoculated from frozen glycerol stocks of E. coli DH5a strains harboring plasmid pUC with SEQ ID NOs: 1-18. These plates are incubated overnight at 30° C. and the resulting bacterial lawn is washed off the plates using two times 2.5 ml pre-warmed EnPressoB medium. The resulting suspension is transferred into a 101 stirred tank bioreactor (Labfors 5, Infors AG, Switzerland) and the cells are cultivated with a set pH of 6.7 and in enzyme controlled glucose limited fed-batch (EnPressoB, Biosilta Oy, Finnland). Before entering the transient phase, the correct time point being determined through previous experiments, cells are harvested by centrifugation with 10000×g and the plasmids harboring the SOI are isolated as described in Example 2.1.

### 2.1 Product Purification

In order to administer the product, key impurities such as proteins, endotoxins, lipopolysaccharides, RNA and chromosomal DNA need to be removed in a scalable and robust process.

Most described plasmid purification protocols consist of three steps: cell lysis, primary purification and concentration through precipitation or filtration followed by a secondary purification step applying one or more polishing chromatography columns.

For isolation, cells harboring the plasmids with the SOI are lysed using alkaline lysis (Bimboim H C and Doly J. (1979) A Rapid Alkaline Extraction Procedure for Screening Recombinant Plasmid DNA. Nuc Acids Res. 7, no. 6: 1513-23, doi:10.1093/nar/7.6.1513.) followed by the above described precipitation. In order to remove impurities in a fast and scalable way, Immobilized Metal Affinity Chromatography (IMAC) is applied. It was shown that free purine bases, as found in RNA and damaged DNA bind, to metal charged resins (e.g. iminodoacetic acid with Cu(II)), while intact plasmid DNA is not retained. In order to release the SOI from the plasmid backbone, the isolated plasmid is cleaved using an appropriate restriction endonucleases (RE), e.g. the RE that was used to ligate the SOI with the replicative plasmid backbone. The SOI is then separated from the replicative plasmid backbone and the RE using a Superose 6 (GE Healthcare, USA) size-exclusion chromatography column.

### 2.2 Storage

The resulting oligonucleotide is stored as described above.

### Example 3

### Product Application

Different means of application of the product are possible:

### 1. Mixing with Beverages

This includes the re-hydration of the lyophilized oligonucleotides on site of the beverage manufacturers and mixing the oligonucleotide with a beverage to a concentration of at least 1 million molecules of oligonucleotides per serving.

### 2. Reversed Spherification

1.5% w/v calcium-lactate hydrate (CAS Number: 41372-22-9) is dissolved in an edible solution (e.g. mango juice, amaretto) and the respective oligonucleotides are added to this solution in varying concentrations. The resulting suspension is dripped into a 0.5% w/v dispersion with a syringe or a JetCutter (geniaLab, Braunschweig, Germany) of sodium-alginate (CAS Number: 9005-38-3) and deionized water and incubated at room temperature for 3 minutes. The resulting oligonucleotide carrier complexes are washed twice with deionized water and stored in the previously used edible solution.

### 3. Mixing with Cosmetics Products

The lyophilized oligonucleotide is resuspended together with a salt of the ethylenediaminetetraacetic acid (EDTA), a common chelating agent in cosmetics used for viscosity control. EDTA is a well known stabilizing agent for oligonucleotides. This suspension is mixed with the cosmetic product in a concentration of at least 1 million molecules/ml. Additionally, omega-3-oleic acid can be added in order to increase skin penetration of the oligonucleotide (Touitou, E, Godin, B et al. (2002) Oleic acid, a skin penetration enhancer, affects Langerhans cells and corneocytes. J Con Rel. 80: 1-7. doi:10.1016/S0168-3659(02)00004-4.).

### Example 4

### In Vitro Digestion of Oligonucleotides

In order to simulate the effect of digestion and oligonucleotide uptake without human trials and without harming endangered species, in vitro gastro-intestinal digestion experiments were performed. These followed the setup as suggested by a leading consortium (Minekus, M. et al. (2014) A standardised static in vitro digestion method suitable for food—an international consensus. Food Funct. 5, 1113-1124. doi: 10.1039/C3F060702J) using an oligonucleotide with SEQ ID NO: 1. In short, oral, gastric and intestinal passage were simulated by emulating their respective chemical and enzymatic compositions. Samples were incubated, as suggested, in each phase for 2 minutes and the oligonucleotides were immediately isolated using the QIAamp DNA Micro Kit (Qiagen, Germany) and stored at 4° C. until quantification.

The integrity of the oligonucleotides after in vitro digestion was determined through realtime PCR. Quantification of full length genes was performed using the Applied

Biosystems 7500 (Thermo Fisher Scientific Inc.) with the gScript™ One-Step SYBR® Green qRT-PCR Kit, ROX™ (QuantaBio Inc.) and oligonucleotide primers (Prim_SEQ1 for [ATGCCCTTGTCCAGTC], Prim_SEQ1 rev[CTAGAAAGCACAGCTG]) annealing (53° C.) at the ends of the full length gene. The use of 1 million oligonucleotide molecules or more resulted in easily detectable amounts of full length oligonucleotide of SEQ ID NO: 1 after passing through the in vitro digestion assay (crossing point (C_{P}) after 23 of 40 cycles). In order to simulate the in vitro passage of a traditional extract, a piece of dried beef meat (“beef jerky”, Bos taurus) was used and oligonucleotides for the corresponding growth factor 1 (GenBank: V00111.1) derived (for [acggctcagg gtccgtgaca], rev [ctaataaaat gaggaaattg]). Dried beef samples were rinsed in deionized, sterile water for 14 hours at 21° C. followed by an incubation with proteinase K (400 ng/mL, P2308 Sigma-Aldrich, USA) and guanidine hydrochloride (5 mol/L, CAS Number: 50-01-1) in 1 mL volume for 12 h at 55° C. The sample was spun down applying centrifugation (15,000 g for 10 min), followed by a phenol/chloroform extraction. Only small amounts of the growth factor 1 gene oligonucleotide could be detected before in vitro digestion (C_{P} at cycle 35 of 40) and no signal above the background threshold after in vitro digestion, clearly underlining the superiority of the synthetic oligonucleotide.

### Example 5

### Effect of Stabilizers on Oligonucleotide Stability

In vitro digestion of oligonucleotides was performed as described in Example 4 with oligonucleotides alone and in combination with various stabilizers. Detection after simulated digested passage at various stages (oral, gastric, intestinal) was again performed by realtime PCR under the conditions indicated in Example 4. Various stabilizers were tested: Stabi1:potassium alginate; Stabi2:hyaluronic acid; Stabi3:poly(ethylene-co-vinyl acetate) Stabi4:poly-L-lactide. As can be seen from FIG. 1, stabilizers had an advantageous effect during a simulated digestive passage since the percentage of detected oligonucleotide (in relation to the initial amount of oligonucleotide) was higher with stabilizer than without stabilizer at all time points.

1-30. (canceled)

31. A formulation comprising at least 1 million oligonucleotides of mammalian, avian, reptilian or plant, non-human DNA, which oligonucleotides have sequences that are at least 80% homologous to one or more of the sequences selected from the group consisting of SEQ ID NOs: 1 to 18 for administration to humans for non-immunological uses.

32. A formulation according to claim 31, wherein the oligonucleotides are present in the form of DNA-carrier complexes comprising oligonucleotides and a suitable physiologically acceptable stabilizer.

33. A formulation according to claim 31, wherein the formulation is a cosmetic formulation or a topical dermatological formulation.

34. A formulation according to claim 31, wherein the formulation is a food formulation.

35. A formulation according to claim 34, wherein the formulation is a beverage food formulation.

36. A formulation according to claim 31, wherein the formulation is a medicinal formulation.

37. A formulation according to claim 36, wherein the sequence of the oligonucleotides is selected from the group consisting of SEQ ID NO: 1.

38. A formulation according to claim 36, wherein the sequence of the oligonucleotides is selected from the group consisting of SEQ ID NO: 2.

39. A formulation according to claim 36, wherein the sequence of the oligonucleotides is selected from the group consisting of SEQ ID NO: 3.

40. A formulation according to claim 36, wherein the sequence of the oligonucleotides is selected from the group consisting of SEQ ID NO: 4.

41. A formulation according to claim 36, wherein the sequence of the oligonucleotides is selected from the group consisting of SEQ ID NO: 5.

42. A formulation according to claim 36, wherein the sequence of the oligonucleotides is selected from the group consisting of SEQ ID NO: 6.

43. A formulation according to claim 36, wherein the sequence of the oligonucleotides is selected from the group consisting of SEQ ID NO: 7.

44. A formulation according to claim 36, wherein the sequence of the oligonucleotides is selected from the group consisting of SEQ ID NO: 8.

45. A formulation according to claim 36, wherein the sequence of the oligonucleotides is selected from the group consisting of SEQ ID NO: 9.

46. A formulation according to claim 36, wherein the sequence of the oligonucleotides is selected from the group consisting of SEQ ID NO: 10.

47. A formulation according to claim 36, wherein the sequence of the oligonucleotides is selected from the group consisting of SEQ ID NO: 11.

48. A formulation according to claim 36, wherein the sequence of the oligonucleotides is selected from the group consisting of SEQ ID NO: 12.

49. A formulation according to claim 36, wherein the sequence of the oligonucleotides is selected from the group consisting of SEQ ID NO: 13.

50. A formulation according to claim 36, wherein the sequence of the oligonucleotides is selected from the group consisting of SEQ ID NO: 14.

51. A formulation according to claim 36, wherein the sequence of the oligonucleotides is selected from the group consisting of SEQ ID NO: 15.

52. A formulation according to claim 36, wherein the sequence of the oligonucleotides is selected from the group consisting of SEQ ID NO: 16.

53. A formulation according to claim 36, wherein the sequence of the oligonucleotides is selected from the group consisting of SEQ ID NO: 17.

54. A formulation according to claim 36, wherein the sequence of the oligonucleotides is selected from the group consisting of SEQ ID NO: 18.

55. A method for producing the formulation according to claim 54, comprising the steps of
(i) providing an oligonucleotide selected from the group consisting of SEQ ID NOs: 1 to 18 in a lyophilized form;
(ii) rehydrating the oligonucleotide in lyophilized form.

56. A method of claim 55, additionally comprising a step of mixing the formulation with cosmetic or food products.

57. Use of the formulation of claim 54 in a personal lubricant.

58. Use of the formulation of claim 54 in a spice mixture.

59. A kit comprising a formulation of claim 54.

60. A formulation comprising at least 1 million molecules of a mammalian avian, reptilian or plant, non-human polypeptide, which polypeptide is encoded by a sequence that is at least 80% homologous to one or more of the sequences selected from the group consisting of SEQ ID NOs: 1 to 18 for administration to humans.


SEQUENCE LISTING (22 sequences)

SEQ ID NO: 1 - DNA, 543 bp, Panthera tigris altaica
atgcccttgtccagtctgtttgccaacgccgtgctccgggcccagcacctgcaccagctggccgccgacacctacaaagagtttgagcgggcgtacatcccggagggacagaggtattccatccagaacgcgcaggctgccttctgcttctcggagaccatcccggcccccacgggcaaggacgaggcccagcagagatccgtgagtggccaggggcgcccggccaaggatcaggggcctccctcgtctgcaggagtcaggccctgggcagccttctccctggggccgagggtgtgcagggaggcagactgggcctctccgtccctcttcccctttttgcaggagctggaagatggcagcccccgggccgggcagatcctgaagcaaacctacgacaagtttgacacaaacttgcgcagtgacgacgcgctgctcaagaactatgggctcctgtcctgcttcaagaaggacctgcacaaggctgagacgtacctgcgggtcatgaagtgtcgccgcttcgtggaaagcagctgtgctttctag

SEQ ID NO: 2 - DNA, 705 bp, Panthera tigris altaica
atgttgagagtattgagcagcgatgagcaccgtgaggcggtacagcaacaaaatatcctcgctgtggagggaacctctagcgaactttgcacggagagtcctacctccaattaccggtgcgaaaccagaggaagtggtagagaccgcggtcaggaccgcgtcaaacggcccatgaacgcattcatggtgtggtctcgtgaccaaaggcgcaaggtggctctagagaatccccaaacgcaaaactcagagatcagcaagcagctggggtaccagtggaaaatgcttacagaagccgaaaaatggccgttcttcgaggaggcacagagactacaggcgctgcaccgagagaaatacccgggctataaataccgacctcgtcggaaggccacgccagaaaaaagtgacaaattgctccccgcagagccctcgtccacactctgtagtcagctgcacgcgggagagaggttgtacgccttcccctacaaggacggctgtacgaaggctgcacactcacgaatgaaggaccagttatactcctcctcggaacccatgagcataaccaactcgctgctggaaccgggacctcacaggacctccacaaccctccaaaacagtcctgaagacttggctctgcagctctccgcggatgctcccctttaccgtaacttagagctgggagtttctgaggcttactttgcatggtga

SEQ ID NO: 3 - DNA, 909 bp, Panthera tigris altaica
atgggggtagctctggagcagttcttcatttttgtagggctgctggcgtgcctggcgtacctggtgaagtgtgtgagattctccaaatgtatttttctgcacttctggaaagttttgccaaggtctttcttgaagtcaatgggacaatgggcagtgatcactggagcgggagatgggattgggaaagcttattcatttgagctagcaagacaaggactcaatgttgtgcttatcagccggacactgaaaaaactacaggccattgctgcggagattgagtgtaccacagggagtagcgtgaagattatacaagcagattttaccaaagacaacatctacgagcatattagagaaaaacttgaaggcttagaaattggaattttagtcaacaatgtcggaatgcttccaaactttctcccgagccacttccttgatacaccagatgaaatccagagtctcatccactgtaacatcacctcagtagtgaagatgacacagctaattctgaaacacatggaatcaaggaggaaagggctcatcttgaacgtttctgccggggtggccttctttccctggccactctactccacgtattcagcttccaaggcttttgtgtgtacgttttccaaagccctgcaagcagaatataaaaggaaaggaatcattatccaggtgctgaccccctatgctgtttcaaccccaatgacaaagtatctaaacaccaacatggtaaccaagactgctgatgagtttgttaaagaatcactgaattatgtcatgattggagacgaaacctgtggctgcctcatccatgaaatcttgcggagcttcctgaacctgatcccatcctgggttttgtacagcagcacattccagaagaaatacatggactacctcaagcacaatacc

SEQ ID NO: 4 - DNA, 1620 bp, Buceros rhinoceros silvestris
atgtctcgccagtgcgctgcaaggacccagagcaaaaccggcttcagtgctgcttctgccttcattccgaacgccagcagcaccagcttctgtgtgcgtcctgcaccccaaggcggaagctgcagtattgcccctgggtatggaaggtttactggaggttttggaagcaggagcctctacagttttggtggatgcaagaggatgtctgtagctggaagaggaggtggcttctatggacctgcaggttttggtgctgggatctcctgtggtttcggtggtgcagttggtggtgccttcgaatttggtggtggtgtgggtggccctggattccctgctgtcccagctgggggcatccgtgaagtctcagtcaatcagagccttctgaaacctctcaacctggagattgaccccaacatccagagtatccgcaaggatgagaaggatcagattcaaaccctcaacaataaatttgcctccttcatcgacaaggtccgattccttgagcaacaaaacaaggtcctggagaccaagtgggcccttctacaagaacaggggaacaaaacggtcagaaacgacatcgagcccctctttgagacctacatcaacaacctcaggaggcagctgagcagcttgctgactgacaaggagaacctggcaggagagctgagcaaggtgcaaagccttgctgaggacttcaagaacaaatacgaagaggagatcaacaggcactcagctgttgagaacgagtttgtggtcctgaagaaggaggtggacgctgcctacatgaataagacggagctgcaagccaggctggagtccctcatggaggagatagatttcctcagagccctctatgaagccgagctgtcccagctgcagacccagatctccgacacctccgtcatcctcaccatggacaacaaccgcagcctggacatggacagcatcattgcggaggtcaaagcacagtacgaggacatcgccaaccggagccgggcagaggctgagtcctggtaccagtccaggtacgaagagctgcaggccacggctggcaggcacggggacgacctccgcaacaccaagcaggagatctctgagctcaaccgccacgtccagcggctgaggtctgaaatcgacagcgtgaagaaacagtgtgccagtttgcagacggccatcgcggatgccgagcagcgtggggagctggctctcaaggatgccagggccaaactggctgagctggagacagccctgcagcaggccaagacagatctggcccggcagctccgcgagtaccaggagctcatgaacgtcaagctggccctggacatcgagatcgcaacctacaggaagctgctggagggcgaggagtgcaggctctctggagaaggtgccaatgcagtgaatatctctgtgaccagaacctctgtaggaacaggatatgggagtggaaactgcctcagcttcggaggcagcagtggcgttggaggtggggtctgtgccggaggaacgggcttcagctctggaagcggacaaggcacagccgggtcatgcgtggtgggtggaaacagctccagtgtgaagtatgtctccaccacctcctccaccaagagatgctattag

SEQ ID NO: 5 - DNA, 2175 bp, Callorhynchus milii
atggaagtggctattttaagcagagttgatcaactagttgatgagaaactaaaggcacatttacctaaggtacgactggtccgcgatgctccgagcaactgcttgtgtcctccaggtcccccaggagaaaaaggaaagcacggtcgaagaggtattccaggaccatctggaccgccaggaagagatggatttccgggaaacaaaggagcattgggtctaccagggagaaggggtctaaagggggaacctggtgaaaagggagctccaggagatccaggcatctctattattggacctcgtggccctccaggtcaaccaggagctagaggctttccaggatttccgggcccgatagggttggatggcaaaaatggcaagatgggtccaaaaggtgaaatgggttcaccaggttcaagaggtgaaaagggtgaaactggtcaaaagggagaaaagggtcatcgtggagaatctggattgcattttggtctgcatggtcaaaagggtgagaagggctcgcgtggagattctggactttcatttacacctcctggattgtctgtgctgctagcagctgacgagcagtatatcaaacgccatcaccacttcaagggtgaaccgggacaagctgataataactgttctccagggcctccaggtccaccagggccctccggacccaaaggacctccagggtattcaggacatccaggggcaaacggattaccagggaaaactggagaagctggaaaaccaggaagagatggaatgaggggcttaactggaatgactggtatgaagggtgattccggacttccaggacgtccaggtgaaaagggtgatccaggtgcttctggaaagtcgggagcaaagggtgatccgggaatgccagggctcccaggaagaaagggagagtttggtgagccagggctgcctggtccaacaggccttcctggacctgtagggccgaagggagataaaggaaacattggacaaaagggagagataggaaggcctggcgttccaggaaagcctggagctaagggcataccaagttcatctgcttctgggttaaagggagagccaggggagattggacctccgggagaaaagggtgaaccaggtcttcgtggtcttcgagggaggagaggtcttaggggtgaaaatgggattccaggtgaagctggaatgcttggagaaaggggtgaaaaaggagatgcaggaatatcaaccaacggaattaaaggggaaccaggttcaactggatctcgtggagctccagggcaaaagggggaccctggccagcctggaccaaggggttcgacaggtcagaagggtgaagcaggaaaaactgaaattatcgattacaatggtaacatcagcgaagccctccagggtcccccaggacttcccggacagccaggccaaaagggtgaaataggtttgccaggtacatcaggacttgatggagaaaagggtcctcgcggtaaaccaggagaggtcggattgcccggtccagaaggcccaacgggcatcgaagggctaaaaggagagccaggaattatgggattcccaggacctaaaggaaacaagggagaagttggccaggcaggttttccgggtctggatggacccacgggtgaaaagggagaaagaggggaacctggattgcaaggtccacctggcttgccgggacctgttggtcttcctggaacacctggagatagaggagaaaagggtgatggggttgttggtccagaagggccacttggtccacagggaccaccaggcccaattggactccaggggctttcaggagcaaagggtgatttaggatcggttggtcttaaaggtgacagaggtcctttgggattgcctggtgtaccaggcccagcaggttcacaagggccagcaggaccaaaaggagaaaggggtgataaaggagatgctggacctccaggaccaattggaccacagggaataactggatcactaggaccaaaaggagaaaaaggaggccggggggagaagggaaagagaggttctaaagggcctaaaggggataagggagaccaaggagtgcctggattagatgcaccttgcccattgggtgaagatggcttacctgtacaaggctgttggaataaaggtcagacaccatggctaaatgccaaatag

SEQ ID NO: 6 - DNA, 1122 bp, Rattus norvegicus
atgcctggatggagctgcctggtgacaggagcaggagggtttgtgggccagaggatcatccggatgttggtgcaggagaaagaactgcaggaggtcagggctctggacaaagtcttcagaccagaaaccaaggaggaattctctaagctgcagacaaaggccaaggtgacaatgttagaaggagatattctggatgcccagtacctgaggagagcgtgccagggcatctctgttgtcatccacacggcttctgtcatggatttctcacgtgtcctacccaggcagaccatcctagatgtcaatctgaaaggtacccagaacctattggaggccggtatccacgctagtgtgccagccttcatctactgcagcacagttgacgttgcagggcccaactcctacaagaagaccatcctgaatggccgtgaggaagagcatcatgaaagcacatggtcaaatccatacccatacagcaaaaagatggccgagaaggcagtgctggcagccaatgggagcatcctgaaaaatggtggcacactgcatacttgtgccttaaggcccatgtacatttatggggagagaggtcaattcctttctagaataataattatggccctcaaaaataagggtgttctgaacgttactggcaaattctccatagtcaatccagtgtatgtaggcaatgtggcctgggcacatattctggctgccaggggccttcgagaccccaagaagtcacaaaatatccaaggacagttctactatatctcagatgacacccctcaccaaagctatgatgatttaaattgcaccctgagcaaggaatggggcctccgccttgattccagctggagccttcctctgcccctgctctactggcttgccttcctgctggaaactgtgagcttcctgctgcgtccattttacaactataggccaccctttaactgccacttggtcacactgtcaaatagcaagttcactttctcctacaagaaagctcagagagatctgggctatgagccacttgtcagctgggaggaagccaagcagaaaacctcggagtggatcgggacactagtggagcagcacagggagacactggacacaaagtctcagtga

SEQ ID NO: 7 - DNA, 1483 bp, Ovis aries
ggtcatggagggcgcgtttgcggcaaactggagcgcggaggcggtcaacgggagcgcggcgcctccgggaaccgagggcaatcgcactgccgggccgccacagcgcaacgaggccctggcgcgggtggaggtggccgtgctgtccctcatcctgttcctggcgctgagcggcaacgcgtgcgtgctgctggcgctgcgcaccacgcgccacaagcactcgcgcctcttcttcttcatgaagcacctgagcatagccgacctggcggtggcggtgttccaggtgctgccgcagcttctgtgggacatcacgttccgtttctacgggcccgacctgctctgccgcctcgtcaagtacctgcaggtggtgggcatgttcgcgtccacctacctgctgctgctcatgtcgctcgaccgctgcctggccatctgccagccgctgcgctcgctgcgccgccgcaccgaccgcctggcggtactcgccacctggctcggctgcctggtggccagcgcgccgcaggtgcacatcttctcgctgcgcgaggtggccgacggcgtcttcgactgctgggccgttttcattcagccctgggggcccaaggcctacatcacgtggatcacgctcgccgtctacattgtgccggtcatcgtgcttgccgcctgctacggccttatcagcttcaagatctggcagaacttacggctcaagacggaggcggcggcggcagaggcttccgcgggggctgagggcgcggcggcagactgcgcggggcgcgcggctctggcccgcgtcagcaacgtcaagctcatctctaaggctaagatccgcacggtcaagatgaccttcatcgtcgtgctggccttcatcgtgtgctggacgccattctttttcaagcagatgtggagtgtctgggatgccgatgcgcccaaggaagcctcggctttcatcatcgccatgcttctggccagcctcaacagctgctgcaacccctggatctacatgctcttcacgggccacctcttccaagaccttgtgcagcgcttcctctgctgctcattccgccgcctgaaaggcagccagctgggggagacgagcgtcacgaaaaagatccattcgtacacctttgtcctgagtcggcacagctccagccagagaagctgctcgcagccatccacggtgtgactgatcggccaggggccctccttggcttcgactgtgcgacagcagacagtctggctcttggtggctgtgtacatgtgtgtacaaggtaccttccagtttgtaccccctgcaccttgggcagctgagttgggtgggaagcaacccacgggaagatggtagggtgactcaaccatcaagccaagccccagatcttctgtgggccccatgggtactcctgccctgaccccactgctgccttcctttaacagtaagtcccctacataagaatgagttctgttctgagaaaaaaaaaaaaaaaaaaaa

SEQ ID NO: 8 - DNA, 1818 bp, Pan troglodytes
atgagctggggcacggagctgtgggatcagttcgacagcttagacaagcatacacaatggggaattgacttcttggaaagatatgccaaatttgttaaagagaggatagaaattgaacagaactatgcgaaacaattgagaaatctggttaagaagtactgccccaaacgttcatccaaagatgaagagccacggtttacctcgtgtgtagccttttttaatatccttaatgagttaaatgactatgcaggacagcgagaagttgtagcagaagaaatggcgcacagagtgtatggtgaattaatgagatatgctcatgatctgaaaactgaaagaaaaatgcatctgcaagaaggacgaaaagctcaacaatatcttgacatgtgctggaaacagatggataatagtaaaaagaagtttgaaagagaatgtagagaggcagaaaaggcacaacagagttatgaaagattggataatgatactaatgcaaccaaggcagatgttgaaaaggccaaacagcagttgaatctgcgtacgcatatggccgatgaaaataaaaatgaatatgctgcacaattacaaaactttaatggagaacaacataaacatttttatgtagtgattcctcagatttacaagcaactacaagaaatggacgaacgaaggactattaaactcagtgagtgttacagaggatttgctgactcagaacgcaaagttattcctatcatttcaaaatgtttggaaggaatgattcttgcagcaaaatcagttgatgaaagaagagactctcaaatggtggtagactccttcaaatctggttttgaacctccaggagactttccatttgaagattacagtcaacatatatatagaaccatttctgatgggactatcagtgcatccaaacaggagagtgggaagatggatgccaaaaccacagtaggaaaggccaagggcaaattgtggctctttggaaagaagccaaagccacagtccccacccttaacccctactagtttattcacatccagtactcctaatgggtcccagtttctcacattctccattgagcccgtgcattattgtatgaatgaaataaaaacagggaagcccagaattccttctttcagaagcctcaaaagagggtggtcggtgaagatgggcccagcactagaagatttcagtcatctgccaccagaacagagacgtaaaaaactacagcagcgcattgatgaacttaacagagaactacagaaagaatcagaccaaaaagatgcactcaacaaaatgaaagatgtatatgagaagaatccacaaatgggggatccagggagtttgcagcctaaattagcagagaccatgaataacattgaccgcctacgaatggaaatccataagaatgaggcttggctctctgaagtcgaaggcaaaacaggtgggagaggagacagaagacatagcagtgacataaatcatcttgtaacacagggacgagaaagtcctgagggaagttacactgatgatgcaaaccaggaagtccgtgggccaccccagcagcatggtcaccacaatgagtttgatgatgaatttgaggatgatgatcccttgcctgctattggacactgcaaagctatctacccttttgatggacataatgaaggtactctagcaatgaaagaaggtgaagttctctacattatagaggaggacaaaggtgacggatggacaagagctcggagacagaacggtgaagaaggctacgttcccacgtcatacatagatgtaactctagagaaaaacagtaaaggttcctga

SEQ ID NO: 9 - DNA, 378 bp, Sus scrofa
atggccggacccagccttgcctgctgcctgctcggcctcctggcgctgacctccgcctgctacatccagaactgccccctgggcggcaagagggccgtgctggacctcgacgtgcgcaagtgcctcccctgcggccccgggggcaaaggccgctgcttcgggcccagcatctgctgcggggatgagctgggctgcttcgtgggcacagccgaggcgctgcgctgccaggaggagaactacctgccgtcgccctgccagtccggtcagaaaccttgcgggagcgagggccgctgcgccgccgccggcatctgctgcaaccctgacggctgccgcttcgaccccgcctgcgaccccgaagccaccttttcccagcgctga

SEQ ID NO: 10 - DNA, 3123 bp, Gallus gallus
atgagggtatcaataccagctgtggcagtgtgggggagaattgctccctctcacagcatcactgccattatgattacagatgaccagcagactattgttacaggaagtcaagaggggcaaatatgtctctgggatctttcatcagaattaaagatttcatctaaagaaattctctttggccatactgcttctgtaacttgtttggcaaaagcaagagagtttgagaaacagccatatgtagtaagtgctactgaaaatggggagatgtgtgtttggaatgtcactagtggactgtgcattgagaacacaaagcttccttataggcatacagcaatctattattaccattgctcgttccgaatgacgggtgaaggctggcttctgtgttttggagaatatcaggatgttcttattattgatgctaagactttgggtgttctgcacactttgtcttctcagtcttctgattggataaattgtatgtgtattgttcactctgcaagaattcaagaagactcattggttgctgtgtctgtaactggagttcttaaagcatgggacctatcttcatctttgaccagcatacaggagaggcaaaatgtgtgtgaaaaggaatcaaagtctctgggctgtacaaactgccgagcagtacggttttgcacttacacagaaagactccttctggttgtgtcttccatatgctggcaggtctatgattattgtgatttctccttgctgtgtactgagttctgtaagagtggtcagtgctttgctggtggagaagtactagcagcttacaggcttatcatctggacagaagatggttacagttacatctaccagctgctaaacagtggactttccaaaagcatatatcctgcagatgggaaggtgctgaaagaaactgtttatcctcatttactctgctttactgatgtgaaggaaaataagagtttcccttttgtcatggggtttatgaatgaaaggaaggagcctttctataagattcttttttctggtgaagcttcaggaagaatcactctgtggcatgttcctgatgttcctgcatcaacatttgatggttcgccacaagaaatcccaatttcaacaacatggacacttcaggataattttaataaagcccattcaatggcagagggcattatagatcatctttgtgattctaaagatggaattgaaaatgcagttgtgacttcttccgtgtacatacccagtctggataagctcgtgtgtggatgtgaaaatggggaaattttcataacgccaggtctagaaactgcaagagcaagacttctagaaaatgtgtctttgaaaggtaatctacctcataaagttttgaatggtcatagtagcagagttacatgtttactttatccacatggtaattcagtaaggtttgatcccagctggctgttatcagggggccaggattctactgtgatctgttgggatatatttactggaaaaatcttgcaccgatttagtctgcaatctgggccagtgacagagctattgagatctccagaaaactgcaaattgcagtatcatagcgtagtgtgctgcgtgtgcagtgatcactcagtagcagttctacaccttcagaagaaagtttgtctcttacatgccagaaagcatctgtttcctgtgaagaagataaaatttgaccccatggaaggtctgctaattattgggtgtgaagatgattcggtttatgtttgggaaattgaaacaggcacactggaacgacatgaaacaggtgaaatagcaaaagcaatcctttcttccattggagattcagaatacttagtggtggattctctacatcctgtatctcaggagtcaaaaagaaataaaaatgttcaatacaaatcctccagctcttataagcgtggtatgatctcttacaatcttactcagacagaaaaatcttcagataaggtaatggataacagccttattcagcaaccctttactgttttaccagttaagccaaaatggaacgatgcaaactttcatgttctcttatttgatctggaaaaacttgtggatcttctgctgtcgtatcaaccctgtggattaaagtcatcaaactcattccacaactatgacactctagaaagagccaaaagtactactgagaaaagagcgctgacattaaaaagaaataaaactgctgggttcacattgtcagtggatggacaagtagatactgtttatgcaaaccaagtcaatagagattctaacacaattaggcctttggaagaaagtggtggcataaaaaggcagaaaaaaatgaagagttcaagaaagactaaaatgcagccatcaggaaacattgatgtgaacatcaccacagatacagctaaactgcttttgtcatgtctcttaccctggggtgtagataaagaaatagatgatctttgtgttgggcaattagatatcttaaggctgcagtgtccggttgcttttggacttgtgtcaaatgaacgccacctctcactgatgttgccgggatggaaatgtactgatggtggtgtgctagaagagcacactttaagcaacttgttttcaaaaagagtgcttgatttatcaaacaagtatcttgctgcaactgaaggacgtactggtgagagagatggaacagtggataatgtttatggaacaaagggtgcagaaataatttttttcttattaagcagaatagttttaatcaacagagtttttaacatgccttcagcagatacgagtcaaattgaaagcctacagcaaccagaatctgttcacaacaagtggcaaaatatggaagctgccatatcttcatttcccagtttttatggagagttaccacgcaataaaagtagacgacattcttctgattttgggaatgcttcactgctgaaactcatttgctgttggagtgaccaatctataaagattattgaggcaatgcaagcagtgctgttggcagaagttcagaggactatgacaaccttgagaaagacagcagtctgcagggagccattggctacagtagagaatggaaatggtagctgtgacttatattgtcttggacagttgggaagccagaccaatctgccattt

SEQ ID NO: 11 - DNA, 669 bp, Varanus comodovarensis
agttgcatgaaagctgccccaatgaaagaagttagcctcagaggacaaggcagcttggcttatccaggtctccggacacagggaaatctggagagcatgggcgggcccaatgatgccacgcgagggttgacatctttggcagacacttttgagcatgtcattgaggagcttctggacgagcaggaggtcatccagcccagcaaggaaaacaaggatgctgacatgtactcgtctcgggttatgctaagcagccaagtgcctttggagcctccactgctctttctgcttgaggagtacaaaaactacttggatgctgcaaacatgtccatgagggtccggcgccactctgaccccgctcgccgtggggagctgagcgtgtgtgacagtatcagcgagtgggtaacagcggcggacaaaaagactgcagtggacatgtcaggggcaacggtgacagtcctggaaaaagtcccagtacccaaaggtcaactgaagcaatatttttacgagaccaaatgcaactcgaagggttatacgaaagagggctgcaggggcatagacaagaggtactggaactcccagtgccgaactacccagtcttatgtgagagctctcaccatggataataaaaagagagttggatggcgctttataagaatagacacttcctgt

SEQ ID NO: 12 - DNA, 1938 bp, Oryctolagus cuniculus
atgtccgagcgcaaagaaggcagaggcaaggggaaaggcaagaaaaaggacagaggctctggcaagaagcccgcgcccgccgacggcggcccgagcccagctttgcctccccgactgaaagagatgaaaagccaggagtcagctgcaggctccaagctggtgcttcgctgtgagaccagttcagagtactcctctctcaggttcaagtggttcaagaatgggaatgaattaaaccgaaaaaacaagccacaaaacatcaagattcaaaaaaagccagggaaatcagaacttcgcatcaacaaagcatcactggctgattctggagaatatatgtgcaaagtgatcagcaaattaggaaacgacagtgcctctgccaacatcaccattgtggaatccaatgagatcatcactggcatgccagcctcaacagaaagagcatatgtatcttcagagtctcccattagaatatcagtatcaacagaaggagcaaatacttcttcatccacatcgacgtccaccaccgggacaagtcaccttgtcaagtgtgccgagaaggagaaaactttctgtgtgaatgggggcgagtgcttcatggtgaaagacctttcaaatccttcacgatacctgtgcaagtgcccaaatgagtttactggtgatcgctgccaaaactacgtaatggccagcttctacaagcatcttgggattgaatttatggaagcagaagagctctaccagaagagggtgctgaccataactggcatctgcatcgccctgctggtggtcggcatcatgtgtgtggtggcctactgcaaaaccaagaaacagcggaaaaagcttcatgaccggcttcggcagagtcttcggtcagaacggaacaacatggtgaacatagcaaacgggcctcaccaccccaacccacccccagagaacgtgcagctggtgaatcaatatgtgtctaaaaatgtcatctccagtgagcacattgttgagagagaagcagagacatctttttccaccagtcactatacctcgacagctcatcattccaccactgtcacccagactcctagtcacagctggagcaatggacacactgagagcatcatttcggaaagccactctgtaatcatgatgtcatcagtagaaaacagtaggcacagcagcccaacagggggcccaagaggacgtcttaatggcttgggaggccctcgagagtgtaacagcttcctcaggcatgccagagaaacccctgactcctaccgagactctcctcatagcgaaaggtatgtgtcagccatgaccaccccggctcgtatgtcacctgtagatttccacacgccaagctcccccaaatcgcccccttcggaaatgtctccgcctgtgtccagcatgacggtgtccatgccctccatggcagtcagccccttcgtggaagaagagagacccctgcttcttgtgactccaccaaggctccgggagaagaagtatgaccaccatcctcagcaattcaactccttccaccacaaccccgcgcatgagagtaacagccttccccccagccccttgaggatagtggaggatgaggagtacgaaacgacccaggagtatgagccagctcaggagcctgttaagaaactcgctaatagccggcgagccaaaagaaccaagcccaatggccacattgccaacaggtcggagatggacagcaacacaagctctgagaacagtgactcagagagtgaaacagaggatgaaagagtaggtgaagatacgcctttcctgggcatacagaaccccctggccgccagtcttgaggcagtacctgccttccgcctggctgacagcaggactaacccagcaggccgcttctcaacgcaggaagaaatccaggccaggctgtctagtgtaattgctaaccaagaccctattgctgtataa

SEQ ID NO: 13 - DNA, 1746 bp, Bungarus fasciatus
atgccctcctgccagcctggcaagatgcctgcgccgtggccttggtggctccagctcctcctttgcatcccgtcctgtgtagcggttttgcccggccgggcaggcgagctgaaagtgtccacccagactggctcggtgcgtggcctgagcctgccggttctcgacggccacgtctcggccttcctaggcatcccctttgcggagcctcccctgggcaggatgcgcttcctgcgccctgagcccgtcaagccctggcagcacgtcctggacgccacttcctacaagcccgcctgctaccagatggtggacaccagctacccaggcttccagggcacggagatgtggaaccccaaccggggcatgagcgaggactgcctctacctcaacatctgggtgccctccccgcgccccaaggatgcccccgtcctggtctggatctacggagggggcttctacagcggcgctgcctccctggacgtctatgacggccgcttcctgacctacacgcagaacgtcatcctggtctccctcagctaccgtgtgggggccttcggctttttgggcctcccgggaagtccagaggccccgggaaacatggggctgctggaccagcgcttggccctgcagtggatccagaacaacatccatccttttgggggcaatccgagggcggtcaccgtctttggggagagtgccggggccgcttcggtgggcatgcacctgctttccacccagagccgcaccctcttccagcgggccattctgcagagcggcggccccaacgcaccgtgggccaccgtcacgcctgccgaaagccggggccgggcggcccttctgggaaagcagctgggctgccacttcaacaacgactccgagctggtcagttgcttgcgctcgaaaaacccccaggagttgattgacgaggagtggtccgtcctgccctacaagagcattttccgcttccccttcgtcccggtcattgacggggacttctttccagatacccccgaggccatgctgagctcgggcaacttcaaggagacccaggtgttgctgggcgtggtgaaggacgagggctcctacttcctgatctacgggctgcctggcttcagcaaggacaacgagagcctcatcagccgggccgacttcctggaaggggtgcggatgagcgtgccccacgccaacgacatcgccactgatgccgtggtgctgcaatacactgactggcaggaccaggacaacagggagaagaaccgggaggccctggacgacatcgtgggagaccacaacgtcatctgcccggtcgtgcagttcgccaacgactacgccaagcgcaacagcaaggtgtacgcctacctgtttgaccaccgggcctccaacctcctctggcccccctggatgggggtcccccatggctacgagatcgagtttgtctttggattgccgctcaacgacagcctgaactacacgccccaagagaaggagctgagccgcagaatgatgcgctactgggccaacttcgcccggacagggaaccccaccgaccctgccgataagagcggggcctggcccacctacaccgcctcccagccgcagtatgtccagctcaacacccagccgctggccacccaacccagcctgcgggcccagatctgcgccttctggaaccacttcctccccaaactgctgaacgccacagtcgatcccccgcgcgccgatcgcagacgccgttccgcccgggcctaa

SEQ ID NO: 14 - DNA, 1155 bp, Canis canis
atggagcgcgcgctggcggccaactggagcgcggaggcgggcaacggcagcgaggcggccccggcggcgcagggcaaccgcacggccgggcccccgcagcgcaacgaggccctggcgcgcgtggaggtggccgtgctgtgcctcatcctcttcctggcgctgagcggcaacgcgtgcgtgctcctggcgctgcgcaccacgcgccacaagcactcgcgcctcttcttcttcatgaagcacctgagcatagccgacctggtggtggcggtgttccaggtgctgccgcagctgctgtgggacatcaccttccgcttctacgggcccgacctgctgtgccgcctggtcaagtacctgcaggtggtgggcatgttcgcctccacctacctgctgctgctcatgtccctggaccgctgcctggccatctgccagccgctgagggcgctgcgccgccgcgcggaccgcctggccgtgctcgccacgtggctgggctgcctggtggccagcgcgccgcaggtgcacatcttctcgctgcgcgaggtggccgacggcgtcttcgactgctgggccgtcttcatccagccctgggggcccaaggcctacgtcacgtggatcacgctctccgtctacatcgtgcccgtcatcgtgctcgccgcctgctacggcctcatcagcttcaaaatttggcagaacctgcgactcaagacggcggcggcggcggccgcggaggggcgggagggcgcggggcgcgcggccctggctcgggtcagcagcgtcaagctcatctccaaggccaagatccgcaccgtgaagatgaccttcataatcgtgctggccttcatcgtgtgctggacgccgttcttcttcgtgcagatgtggagcgtctgggacgccgatgcgcccaaggaagcctcggctttcatcatagccatgctcctggccagcctcaacagctgctgtaacccttggatctacatgctcttcacgggccacctcttccatgaacttgtacagcgcttcctctgctgctcctccagctacctgaaggggaaccgtccaggggagaccagcgtcagcaaaaagagcaactcatccacctttgtcctgagccaccacagctccagccagaggagctcctcgcagccatccacagtgtga

SEQ ID NO: 15 - DNA, 1833 bp, Bubalus bubalis
atgcaggtccccagccccagcgtgcgcgaggcggcctccatgtacggcaccgcggtggccgtcttcctggtcatcctcgtggctgcgctgcagggctcggcccccgccgagagccccttccccttccacatccccctggaccccgaggggaccctggagctgtcctggaacgtcagctacgcgcaggagaccgtctacttccagctcctggtgcgggagctcaaggctggtgtcctgttcgggatgtcggaccgaggggagctggagaatgctgatctggtggtgctctggactgacagggacggcgcctacttcggggatgcctggagtgaccagaaggggcaggtccacctggactcccagcaggattaccagcttctgcgggcacagaggactccagaaggcctgtacctgctcttcaagaggccttttggcacctgtgaccccaacgactacctcatcgaggacggcaccgtccacctggtgtacggactcctggaggagccgctccggtcgctggaggccatcaacatgtccggcttgcgcacggggctgcagagggtgcagctgctgaagcccagcatcctgcagccggccctgcccacagacacacgcaccatggagatccgcgcccctgacgtcctcatccctggccagcagaccacgtactggtgctacgtgaccgagctcccagacggcttcccccggcaccacatcgtcatgtacgagcccatcgtcaccgagggcaacgaggcgctggtgcaccacatggaggtcttccagtgcgccgccgagttcgagaccatcccccacttcagcgggccctgcgactccaagatgaagccgcagcggctcaacttctgccgacacgtgctggccgcctgggccctgggcgccaaggccttttactacccagaggaagcgggcctggccttcggggggcccggctcctccagatttctccgcctggaagttcactaccacaacccgctggtgataacaggccggcgcgactcctcgggcatccgcctgtactacacggccgcgctgcggcgcttcgacgcgggcatcatggagctgggcctggcgtacacgcctgtgatggccatccccccgcaggagacggccttcgtcctcaccggctactgcacagacaagtgcacccagctggccctgcccgcctcggggattcacatcttcgcctctcagctccacacgcacctgacgggccggaaggtggtcacggtgctggccagggacggccgggagacagagattgtgaacagggacaaccattacagcccgcacttccaggagatccgcatgttgaagaaggtcgtgtctgtccacccgggagacatgctcatcacctcttgcacgtacaacacggaagacaggaggctggccaccgtggggggcttcgggatcctggaggagatgtgcgtcaactacgtgcactactacccccagacgcagctggagctctgcaagagcgccgtggaccctggcttcctgcagaagtacttccaccttgtgaacaggttcaacagtgaggaagtctgcacctgcccccaggcgtctgtccccgagcagtttgcctccgtgccctggaactccttcaaccgcgaggtgctcaaggccctgtacggcttcgcgcccatctccatgcactgcaacaagtcctcggccgtccgcttccagggcgagtggaatcggcagcccctgcctgagatcgtgtccaggttggaagagcccacccctcactgcccggccagccaggctcagagccccgctggccccaccgtgctcaacatcggtgggggcaaaggttga

SEQ ID NO: 16 - DNA, 1352 bp, Equus caballus
cgccgcggacgcggacgggctgctggcggggcgcgggccgggtgcgggcggcggtgcggggacccctggggcggcggcggcgttggcggggggcgtgctgctcatcggcgcagtgctcgcggggaactcgctcgtgtgcgtgagcgtggcggccgagcgctccctgcagacgcccaccaactactttatcgtgagcctggcggccgccgaccccctactcgccctgctcgtgctgcctctcttcgtctactccgaggtccagggcggcgtgtggctgctgagccccggcctctgcgacgcgctcatggccatggacgtcatgctgtgcaccgcctccatcttcaacctgtgcgccatcagcgtggacaggttcgtggccgtggccgtgcccttgagctacaaccgccagagccggggcggccgtcagctgctgctcatcggcgccacgtggctgctgtcggcggcggtggccgcgcccgtgctgtgcggcctcaacgacgctcacggccgcgaccccgccgtgtgccgcctggaggaccgcgactacgtggtctactcatccgtgtgctccttcttcctgccctgcccgctcatgctgctgctctactgggccaccttccggggcctgcggcgctgggaggccgcccgccgcgccaagctgcacggccgtgccccgcaccggaccagcggcccgggccctccggcccccgacgccaccgcgctccggggcccgctgccccccgacgccatcccgccccctgggccacagcccccagacacaacccagctccccagggccatcacgtctcccaacgccatcccgttctccgacgccaatgcgcacccggacgcaacccagctccccagtgtcaccccggcctccgacgccaccccgccccccgacgccaccgcaccccctgacgccaccccgccccctgacgccatcctgtaccccgacgccactgcaccccctgacgccaccctgccccccgacgccatcctgtccccggatgccatcccgtcccccgatgccatcccgtgccccgatgccgtcgcacccctccacgccatcccggccgagcccccgctgcaggcccgcaggaggaggcgcgccaagatcaccggccgggagcgcaaggccatgggggtcctgcctgtggtggtcggggtcttcctgctgtgctggtcgcccttcttcgtcgtgcacatcacgcgggcactgtgtcccacctgcgcggtgtccccgcggctggtcagcgcagtcacctggctgggctacgtcaacagcgccctcaaccccgtcatctacaccgtcttcaacgccgagttccgcaacgtcttccgcaaggccc

SEQ ID NO: 17 - DNA, 522 bp, Haliaeetus leucocephalus
atggatatcaccattcataaccccctgatccgcagacccctgttatcttggttggcaccaagtcgtatctttgaccagatttttggagagcacctgcaggagtcagagctgctccctgcttcccccagcctcagtcccttcctgatgagatcccccattcttcggatgcccagttggctagagacaggactctctgagatgcgactggagaaggacaaattttctgtaaatcttgatgtgaagcatttctcccctgaggagctaaaagtgaaggtgcttggggacatgatagaaattcatgggaaacacgaggagcgccaggatgagcatggctttattgccagggagttcaacaggaaatacagaattccagatgatgtggaccctctgaccataacctcatcgctctctctggatggtgtcctgactgtgagtgcaccaaggaaacaaagtgatgtccccgagcgctctattcctatcacccgtgaagagaagcctgccattgcaggagcccaaaggaag

SEQ ID NO: 18 - DNA, 390 bp, Phyllostachys edulis
atggcgaagaagattgtgcaagcggccaacttgaagcagatcctcaggaggtgctcgagcctggggaggaggcagcaggacttggtgcccaagggccacttcccggtctacgtcggcgagagccggtgccggttcgtcgtgccgatcgcctgcctggagcacccggagttcctggtgctgctgcggaaggccgaggaggagttcgggttcgagcacgacgccgccatcacgctgccctgcaatgaggccgtcttcgaggctgtcctcgcctccttgacgggtccaagctcttccttcttccctgatcttagagaccggctctatagacgaggagaatcgacgtttgaagatcctggtattgcgctctatccagatatcccaagtgattag

SEQ ID NO: 19 - DNA, 16 bp, Artificial Sequence
  Forward primer for amplifying SEQ ID NO1
atgcccttgtccagtc

SEQ ID NO: 20 - DNA, 16 bp, Artificial Sequence
  Reverse primer for amplifying SEQ ID NO1
ctagaaagcacagctg

SEQ ID NO: 21 - DNA, 20 bp, Artificial Sequence
  Forward primer for amplifying growth factor 1 of Bos taurus
acggctcagggtccgtgaca

SEQ ID NO: 22 - DNA, 20 bp, Artificial Sequence
  Reverse primer for amplifying growth factor 1 of Bos Taurus
ctaataaaatgaggaaattg